(19) United States
(12) Patent Application Publication
(10) Pub. No.: US 2021/0354339 A1
(43) Pub. Date: Nov. 18, 2021

(54) APPARATUS FOR MANUFACTURING SKIN CARE PACK

(71) Applicants: AMOREPACIFIC CORPORATION, Seoul (KR); LINCSOLUTION CO., LTD., Siheung-si, Gyeonggi-do (KR)

(72) Inventors: Sung Won YI, Yongin-si (KR); Ji Hoon KIM, Yongin-si (KR); Jae Min LIM, Yongin-si (KR); Jeong Eun SEO, Yongin-si (KR); Yang Gyu CHOI, Yongin-si (KR); Keun Sik CHOI, Siheung-si (KR); Kang Wook LEE, Siheung-si (KR)

(73) Assignees: AMOREPACIFIC CORPORATION, Seoul (KR); LINCSOLUTION CO., LTD., Siheung-si, Gyeonggi-do (KR)

(21) Appl. No.: 17/286,260

(22) PCT Filed: Oct. 18, 2019
(86) PCT No.: PCT/KR2019/013756

§ 371 Date: Apr. 16, 2021

(30) Foreign Application Priority Data
Oct. 19, 2018 (KR) 10-2018-0125574

(51) Int. Cl. B29C 41/00; B05B 9/04; B05B 9/00; B05B 12/00; B29C 41/52; B29C 41/08; B29C 41/46; B29C 41/36; A45D 44/00; A45D 34/04

(52) U.S. Cl. CPC B29C 41/003; B05B 9/0413; B05B 9/002; B05B 12/004; B29C 41/52; B29K 2105/24; B29C 41/46; B29C 41/365; A45D 44/002; A45D 34/04; B29C 41/08

(57) ABSTRACT

One aspect of the present disclosure relates to an apparatus for manufacturing a skin care pack. According to an aspect of the present invention, the apparatus for manufacturing a skin care pack may be provided, the apparatus containing: a housing for providing a working space for forming a skin care pack; a film moving module provided to be movable in one direction within the working space, and having a base on which a film is mounted; a molding machine provided to be movable within the working space in two directions perpendicular to the one direction, and including at least one nozzle module for discharging hydrogel to the base; and a controller for controlling movement of the film moving module and the nozzle module, and controlling discharge of the hydrogel from the molding machine.

### TECHNICAL FIELD

One aspect of the present disclosure relates to an apparatus for manufacturing a skin care pack.

### BACKGROUND ART

A mask pack is a kind of cosmetic which can relatively conveniently and effectively perform skin care related to skin winkle, skin elasticity, gloss or the like by supplying moisture and nutrition to skin.

These mask packs have various forms such as a sheet product of non-woven fabric material to which a liquid such as a skin lotion is applied, a mask pack product which exhibits improved wearing-feeling by having an essence contained within a fabric, such as cotton, a mask pack product which uses hydrogel, or a bio-cellulose mask pack product which uses a natural material. As the mask pack product using hydrogel among these has an advantage that a functional component for skin care is selectively contained or mixed, demand for the hydrogel mask pack is increasing.

Meanwhile, a manufacturer mass-produces and supplies mask packs to the market using a factory automation system which can produce a great number of mask packs for a short time period after determining a product standard based on a face model of a general user for mass production of the mask pack.

The mask packs supplied by mass-production are getting good response in the market because they exhibit their effects beyond a certain level at a relatively inexpensive price. Nevertheless, a user cannot use a mask pack which perfectly fits to his/her own skin due to the limit of mass production system. So, there is a drawback that a user cannot feel enough satisfaction with the mask packs supplied by mass-production.

In view of this background, recently there have been trials to manufacture a user-customized mask pack. Specifically, there is suggested a technology which generates a 3D model of a user face, and produces a mask pack fit to a face shape of a user using it. This prior art is characterized by fabricating a sheet such as non-woven fabric or cotton based on modeled data so as to fit to a user's face, or applying substance for skin care to a specific region of the sheet in view of a face shape of a user.

However, the above-described apparatus for manufacturing a customized mask pack is applicable to a mask pack having a sheet, but cannot be applied to manufacturing a hydrogel mask pack for which demand is increasing recently. That is because hydrogel is in a semi-solid state at a room temperature and thus is required to be heated for forming to a desired shape, which may lead to a drawback that, when hydrogel is heated, its viscosity is decreased and hydrogel leaks from a nozzle through which hydrogel is discharged. That is, with the conventional manner, it is very difficult or substantially impossible to precisely control a discharge timing, a discharge position and a discharge amount of hydrogel in order to manufacture a customized mask pack.

Further, if the heating temperature of hydrogel is lowered in order to prevent this problem, its viscosity enough for forming cannot be acquired, and thus it cannot be avoided that productivity of a mask pack is extremely lowered or quality of the final product becomes very bad.

Korean Patent Application Publication No. 10-2017-0070699 (Published on Jun. 22, 2017) provides “a method for manufacturing a 3D-hydrogel mask”, and however, it is only intended to optimize hydrogel contents, while still having the above-described problem. Thus, it cannot become a substantial countermeasure for producing a hydrogel mask pack.

Additionally, since Korean Patent Application Publication No. 10-2017-0070699 focuses on manufacturing a hydrogel mask pack one by one, there is a problem that the mask pack cannot be continuously mass-produced.

### SUMMARY

Embodiments of the present disclosure, which have been proposed to address the above-mentioned problems, are to provide an apparatus for manufacturing a skin care pack which is capable of rapidly and elaborately manufacturing a skin care pack even while using a hydrogel as a raw material.

In addition, embodiments of the present disclosure are to provide an apparatus for manufacturing a skin care pack optimized to physical characteristics of a user.

Additionally, embodiments of the present disclosure are to provide an apparatus for manufacturing a skin care pack capable of continuously mass-producing skin care packs.

According to an aspect of the present invention, there is provided an apparatus for manufacturing a skin care pack, the apparatus comprising: a housing providing a work space for forming a skin care pack; a film moving module which is provided to be movable in one direction within the work space and has a base on which a film is seated; a former which is provided to be movable in two directions perpendicular to the one direction within the work space, and includes at least one nozzle module for discharging hydrogel toward the base; and a control unit which controls the movement of the film moving module and the nozzle module, and controls the discharge of the hydrogel from the former.

Further, there is provided an apparatus, further comprising a material supply device for supplying the hydrogel to the nozzle module.

Further, there is provided an apparatus, wherein the material supply device comprises: a storage tank in which the hydrogel is stored; and a compressor providing pressure to the storage tank to move the hydrogel stored in the storage tank to the nozzle module.

Further, there is provided an apparatus, further comprising a pressure sensor provided inside the storage tank, wherein the control unit controls the pressure inside the storage tank based on pressure measured by the pressure sensor, and controls the amount of the hydrogel moved from the storage tank to the nozzle module.

Further, there is provided an apparatus, further comprising a tube which connects the material supply device and the nozzle module, so that the hydrogel is transferred from the material supply device to the nozzle module.

Further, there is provided an apparatus, further comprising a temperature sensor which is provided between the outer circumferential surface and the inner circumferential surface of the tube to measure the temperature of the hydrogel transferred in the tube.

Further, there is provided an apparatus, further comprising a first heating unit which surrounds at least a portion of the tube to heat the hydrogel that is moved from the material supply device to the nozzle module.

Further, there is provided an apparatus, wherein the material of the tube is Teflon.

Further, there is provided an apparatus, wherein the nozzle module comprises: a cartridge which is connected to the tube and into which a hydrogel is introduced from the tube; a piezo jet spraying nozzle which sprays the hydrogel introduced into the cartridge; and a second heating unit which surrounds at least a portion of the cartridge and the piezo jet spraying nozzle so as to heat at least a portion of the cartridge and the piezo jet spraying nozzle.

Further, there is provided an apparatus, wherein a hole is formed in the second heating unit, so that the amount of the hydrogel remaining in the cartridge can be checked.

Further, there is provided an apparatus, wherein the film moving module comprises: a first platform having a base on which the film is seated; a second platform which is disposed under the first platform, and has a guide part guiding the first platform to reciprocate in a Y-axis direction; and a Y-axis drive unit reciprocating the first platform in the Y-axis.

Further, there is provided an apparatus, wherein the former comprises: an X-axis moving module which moves the nozzle module in an X-axis direction; and a Z-axis moving module which moves the nozzle module in a Z-axis direction.

Further, there is provided an apparatus, wherein the X-axis moving module comprises: an X-axis guide plate which guides the nozzle module in the X-axis direction; a universal adapter plate supporting the X-axis guide plate; and an X-axis drive unit which is coupled to the universal adapter plate, and reciprocates the nozzle module in the X-axis.

Further, there is provided an apparatus, wherein the Z-axis moving module comprises: a Z-axis guide plate guiding the nozzle module in the Z-axis direction; a support member which supports the Z-axis guide plate; and a Z-axis drive unit which is coupled to the support member, and reciprocates the nozzle module in the Z-axis.

Further, there is provided an apparatus, further comprising: a residual pressure valve provided on a first connection pipe connecting the storage tank with the compressor; and a change valve provided on a second connection pipe connecting the compressor with the tube, wherein the control unit controls opening and closing of the residual pressure valve and the change valve, so that air discharged from the compressor is supplied to the storage tank, or air discharged from the compressor is supplied to the tube.

The apparatus for manufacturing a skin care pack according to the embodiments of the present disclosure can rapidly and precisely manufacture a skin care pack even while using hydrogel as a raw material.

Additionally, the apparatus for manufacturing a skin care pack according to embodiments of the present disclosure can manufacture a skin care pack optimized to physical characteristics of a user.

Additionally, the apparatus for manufacturing a skin care pack according to embodiments of the present disclosure can continuously mass-produce skin care packs.

### BRIEF DESCRIPTION OF THE DRAWINGS

FIG. 1 is a perspective view of an apparatus for manufacturing a skin care pack according to an embodiment of the present disclosure.

FIG. 2 is an exploded perspective view of the apparatus for manufacturing a skin care pack shown in FIG. 1.

FIG. 3 is a perspective view showing the film moving module according to an embodiment of the present disclosure.

FIG. 4 is a perspective view showing a former according to an embodiment of the present disclosure.

FIG. 5 is an exploded perspective view of the former shown in FIG. 4.

FIG. 6 is a perspective view showing a material supply device according to an embodiment of the present disclosure.

FIG. 7 is a schematic diagram briefly showing a cross section of a tube connecting a material supply device and a nozzle module according to an embodiment of the present disclosure.

FIG. 8 is a perspective view showing a nozzle module according to an embodiment of the present disclosure.

FIG. 9 is a schematic diagram showing a path through which air coming from a compressor is moved.

### DESCRIPTION OF EMBODIMENTS

Hereinafter, specific exemplary embodiments of the present disclosure will be described in detail with reference to the drawings.

Additionally, it is noted that in the description of the disclosure, the detailed description for known related configurations or functions may be omitted when it is deemed that such description may obscure essential points of the disclosure.

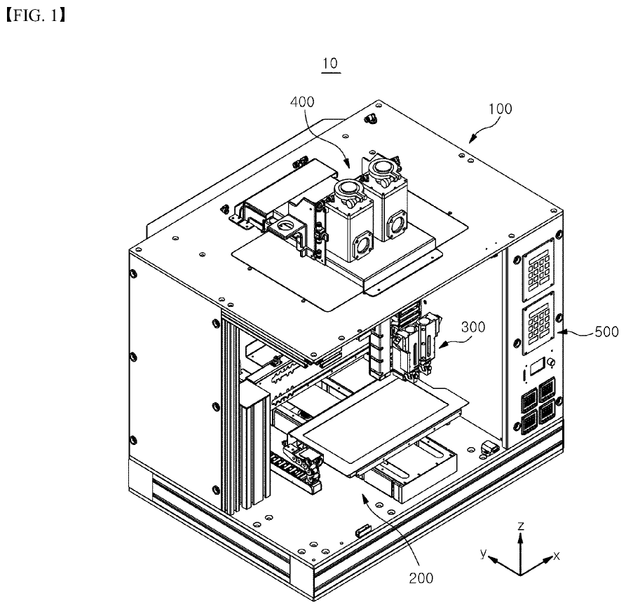

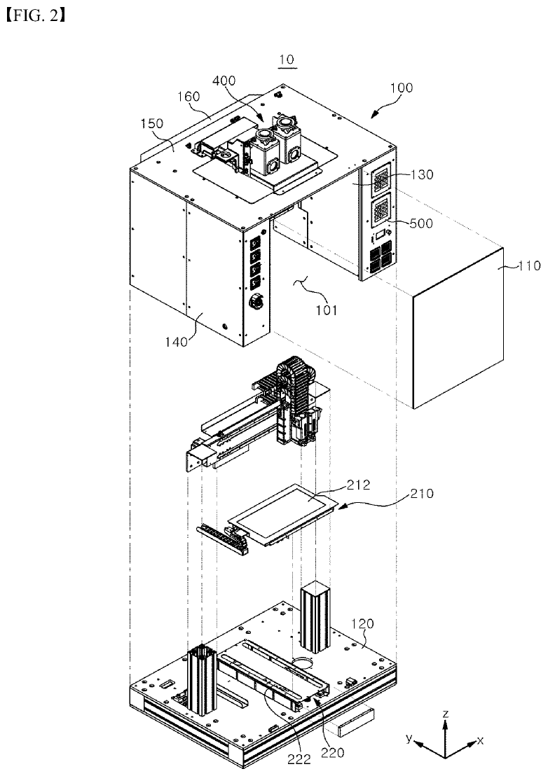

FIG. 1 is a perspective view of an apparatus for manufacturing a skin care pack according to an embodiment of the present disclosure, and FIG. 2 is an exploded perspective view of the apparatus for manufacturing a skin care pack shown in FIG. 1. Additionally, in FIG. 1, a portion of a housing is shown in a transparent manner for convenience of description, so that structures inside the housing may be readily seen.

Referring to FIGS. 1 and 2, the apparatus 10 for manufacturing a skin care pack according to an embodiment of the present disclosure may include a housing 100, a film moving module 200, a former 300, a material supply device 400, and a control unit 500.

In an embodiment of the disclosure, as an apparatus that is intended to manufacture a skin care pack for which a raw material is hydrogel and which may be attached to the skin for use by a user, the apparatus 10 for manufacturing a skin care pack may manufacture a skin care pack based on modeling data for any body part such as a face, a hand, an arm, a foot, a leg or the like of the user. In the embodiment of the disclosure and the description blow, the skin care pack is described by way of example as being a mask pack which is for attachment to a user's face, but the technical idea of the disclosure is not limited to this.

The housing 100 includes a bottom portion 120, a right wall portion 130, a left wall portion 140, a ceiling portion 150, and a rear portion 160 to provide a working space 101, and includes a door 110 for selectively opening and closing the work space 101. Here, the door 110 may employ a transparent material, such as glass, so that the work space 101 can be seen, and the control unit 500 to be described later may be located inside the right wall portion 130 or the left wall portion 140.

The film moving module 200 is disposed on the bottom portion 120 of the housing 100, and a film is located on the film moving module 200. In addition, raw material is applied on a film located on the film moving module 200 to form a mask pack.

In an embodiment of the disclosure, the raw material has the property of a semi-solid or gel substance at room temperature, but when the raw material is heated, its viscosity is lowered to a certain level and may have the property of a liquid substance. For example, the raw material may maintain a viscosity ranging from 120 CPS to 2,500 CPS at a temperature ranging from 70° C. to 95° C., which enables it to be discharged through a nozzle. However, if it is heated to 100° C. or higher, a problem that water which is one of components of the raw material is evaporated may occur. So, it is preferable to set the temperature of the raw material to be 95° C. or lower for the purpose of safety. Specifically, the raw material may be any one of hydrogel, gel type synthetic resin, and a material which contains a raw material for a functional cosmetic in polymer, and in the present embodiment, it is described by way of example as being hydrogel.

The former 300 is located above the film moving module 200, and is disposed in the work space 101 of the housing 100. The former 300 may discharge onto the film moving module 200 a raw material for forming a mask pack. For example, the former 300 serves to form a mask pack on the film by discharging a raw material onto the film that is mountable on or separable from a base 212 of a first platform 210.

Here, the raw material applied on the film is in a heated condition. For example, the raw material may be in a heated condition of about 90 degrees Celsius. The heated condition may be controlled by the control unit 500.

The material supply device 400 is located outside the housing 100, stores the hydrogel and supplies the hydrogel to the nozzle module 310. In this way, it is possible to continuously supply the hydrogel to the nozzle module 310 from the material supply device 400 disposed outside the housing 100, so that a continuous process and mass production can be performed.

The control unit 500 may be electrically connected to the film moving module 200, the former 300, the material supply device 400, and a device attached to a tube 600 through various electric wire cables, and may control the electrical configuration of the film moving module 200, the former 300, the material supply device 400, and the device attached to the tube 600.

The control unit 500 may include an input/output display device (e.g., touch screen), an electronic circuit device connected thereto and a power supply device.

The control unit 500 may further include a USB port (not marked) for inputting and outputting external data. The control unit 500 may control the operation of the film moving module 200, the former 300, and the material supply device 400, receive setting values required to forming a mask pack and customized modeling CAD data for each user, and display an operation state. The control unit 500 may be disposed in the right wall portion 130 and the left wall portion 140 of the housing 100.

Here, the control unit 500 may calculate or set a movement path of the former 300, a discharge speed, a discharge amount, a discharge timing of hydrogel or the like for forming a mask pack. That is, the control unit 500 controls the discharge of hydrogel at the nozzle module 310. Basic data for this may be transmitted from the outside through wired/wireless data communication, or through a storage means such as a USB or the like.

In addition, the control unit 500 may control the temperature of the hydrogel discharged from the former 300 to the film moving module 200, the amount of hydrogel moving from the material supply device 400 to the nozzle module, and the like.

In addition, the controller 500 may control the temperature of the hydrogel stored in the material supply device 400, or control the amount of the hydrogel discharged to the former, based on a temperature sensor and a pressure sensor provided to the material supply device 400 to be described later.

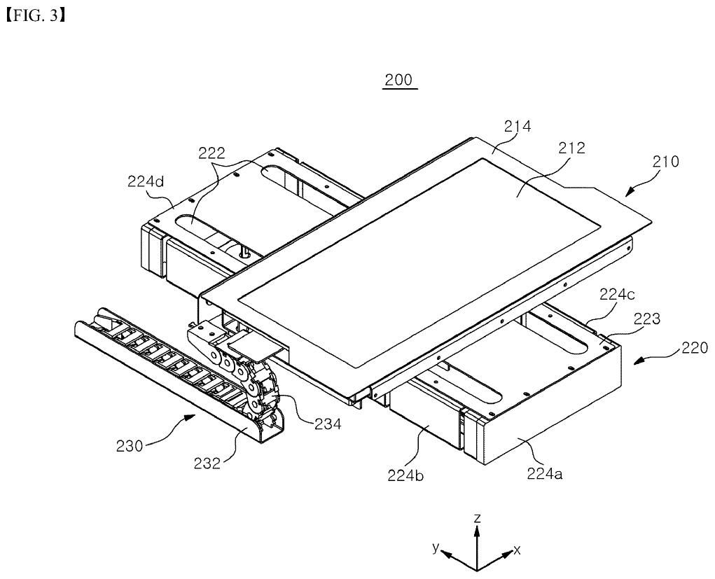

FIG. 3 is a perspective view showing the film moving module 200 according to an embodiment of the present disclosure.

Referring to FIG. 3, the film moving module 200 may include a first platform 210 located on the bottom 120 of the housing 100 and having a base 212 on which the film is located, a second platform 220 having a guide portion 222 for guiding the first platform 210 to move linearly, and a Y-axis drive unit (not shown) for reciprocating the first platform in the Y-axis.

The first platform 210 includes the base 212 on which a film is located. In addition, the first platform may include a perimeter portion 214 surrounding the base 212. Here, the thickness of the perimeter portion 214 may be the same as the thickness of the film. For example, the thickness of the perimeter portion 214 may be 0.2t (here, t is a natural number).

The first platform 210 may perform high-speed reciprocating motion in the Y-axis direction in correspondence to the high-speed injection of the hydrogel from the piezo jet nozzle 316 to be described later. For example, the moving speed of the first platform 210 may be faster than that of the nozzle module 310 moving in the X-axis and Z-axis directions.

left-right direction is referred to as the X-axis direction, and the front-back direction is referred to as the Y-axis direction, and the Z-axis direction is described as a direction perpendicular to the X-axis and Y-axis directions. Further, this direction setting is only an example, and a direction indicated by each axis direction may be set differently according to an exemplary embodiment. In addition, while each component of the present embodiment is described by way of example as being driven based on an orthogonal coordinate system, the technical idea of the present disclosure is not limited to this, but each component may be driven based on a polar coordinate system.

Materials of the base 212 and the perimeter portion 214 may be stainless steel (e.g., SUS 430 series material). Here, stainless steel has a rust-preventive function, and a film holder (not shown) may be attached by a magnetic force based on properties of SUS 430 series material.

In addition, a heater (not shown) for heating the hydrogel discharged to the base 212 may be included under the base 212. Here, the heater may be a pad type or film type heating element installed under the base 212.

In addition, a heater (not shown) transfers heat to the base 212 to prevent the hydrogel discharged on the base 212 from hardening or to heat the hydrogel, and as an example, the temperature of the base 212 may be maintained at 20 degrees Celsius. Here, the temperature of the base 212 may be lower than that of the hydrogel discharged from the nozzle module 310.

An electrostatic removing device (not shown) installed on the upper part of the base 212 may be further provided on the rear surface part 160 of the housing 100 serving as a support base thereof. Here, the electrostatic removing device may be constituted by an ion gun and a foreign substance blowing device, so that, by removing electrostatic from the film by irradiating ions toward the film, it is possible to prevent dust or micro foreign matters from being attached to the film, and relatively big foreign matters can be removed by a fluid force of the blowing device. Accordingly, a hydrogel mask pack without foreign matter or dust may be formed on the film.

Meanwhile, there is also a case where a mask pack is constituted by two segments correspondingly to upper and lower portions of a user's head. In order to manufacture such mask pack with ease, it may have a base 212 partitioned into a plurality of parts by the perimeter portion 214, and separate films may be placed on the plurality of parts of the base 212, respectively.

The second platform 220 may be located under the first platform 210, and include a guide part 222 along which the first platform is linearly reciprocated in the Y-axis direction. The second platform 220 may be formed with four planar members 224a, 224b, 224c, and 224d forming side surfaces, and one top surface member 223 forming a top surface. Here, the guide part 222 may be configured by forming a hole in the top surface member 223. By forming the hole parallel to the Y-axis, the first platform 210 may be linearly reciprocated in the Y-axis direction by the hole.

The Y-axis drive unit (not shown) reciprocates the first platform 210 in the Y-axis, and may be disposed under the second platform 220. Here, the Y-axis drive unit may be a linear motor.

In addition, the film moving module 200 may include an auxiliary guide part 230 for assisting the reciprocating movement of the first platform 210 in the Y-axis. The auxiliary guide part 230 may be moved along an auxiliary guide rail 232 parallel to the Y-axis direction, and include a Y-axis auxiliary chain 234 connected to the first platform 210.

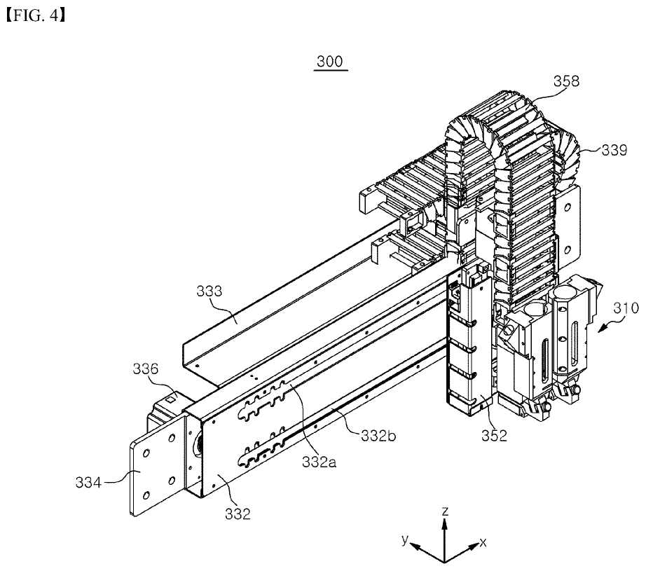

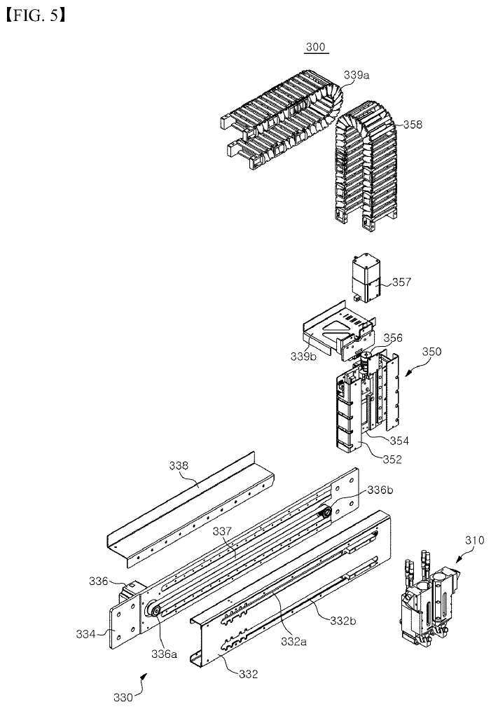

FIG. 4 is a perspective view showing the former 300 according to an embodiment of the present disclosure, and FIG. 5 is an exploded perspective view of the former 300 shown in FIG. 4.

Referring to FIGS. 4 and 5, the former 300 may include the nozzle module 310 for discharging the hydrogel to the base 212 (see, FIG. 2), and an X-axis moving module 330 for moving the nozzle module 310 in the X-axis direction, and a Z-axis moving module 350 for moving the nozzle module 310 in the Z-axis direction.

The nozzle module 310 is connected to the tube 600 to be described later, and discharges the hydrogel to the base 212, and a detailed description of the nozzle module 310 will be presented below.

The X-axis moving module 330 is configured to reciprocate the nozzle module 310 in the X-axis direction. For example, the X-axis moving module 330 may include an X-axis guide plate 332 for guiding the nozzle module 310 in the X-axis direction, a universal adapter plate 334 for supporting the X-axis guide plate 332, and an X-axis drive unit 336 coupled to the universal adapter plate 334 to reciprocate the nozzle module 310 in the X-axis. Here, the universal adapter plate 334 may be directly supported on the housing 100, or may additionally include a support plate 338 for connection thereof to the housing 100.

In addition, the X-axis moving module 330 may include an X-axis auxiliary chain 339a which assists the movement of the nozzle module 310 in the X-axis direction, and an X-axis guide rail 339b which guides the movement of the X-axis auxiliary chain 339a. Here, the X-axis guide rail 339b may be provided at the rear surface of the Z-axis moving module 350 to be described later, and at the top of the support plate 338.

One or more holes 332a and 332b may be formed in the X-axis guide plate 332 in the X-axis direction, so that the nozzle module 310 may be moved in the X-axis direction while being guided by the holes 332a and 332b.

The universal adapter plate 334 may be provided with a plurality of mounting holes to improve a degree of freedom of mounting the X-axis guide plate 332 thereto.

The X-axis drive unit 336 may be coupled to the rear surface of the universal adapter plate 334, and there may be a plurality of the X-axis drive units. For example, there may be two X-axis drive units 336, and a chain 337 may be connected between both X-axis drive unit heads 336a and 336b. In this case, by driving the X-axis drive unit 336, the chain 337 connected to the X-axis drive unit heads 336a and 336b may be rotated, so that the nozzle module 310 may be moved in the X-axis direction.

The Z-axis moving module 350 is configured to reciprocate the nozzle module 310 in the Z-axis direction. For example, the Z-axis moving module 350 may include a Z-axis guide plate 352 guiding the nozzle module 310 in the Z-axis direction, a support member 354 supporting the Z-axis guide plate 352, and a Z-axis drive unit 356 coupled to the support member 354 to reciprocate the nozzle module 310 in the Z-axis. Here, since the support member 354 is movably coupled to the X-axis guide plate 332 of the X-axis moving module 330, the nozzle module 310 is movable in the Z-axis and the X-axis.

In addition, around the Z-axis drive unit 356 a Z-axis drive unit protective cover 357 may be included for surrounding the Z-axis drive unit 356 and protecting the Z-axis drive unit 356.

In addition, the Z-axis moving module 350 may further include a Z-axis auxiliary chain 358 which assists the movement of the nozzle module 310 in the Z-axis direction.

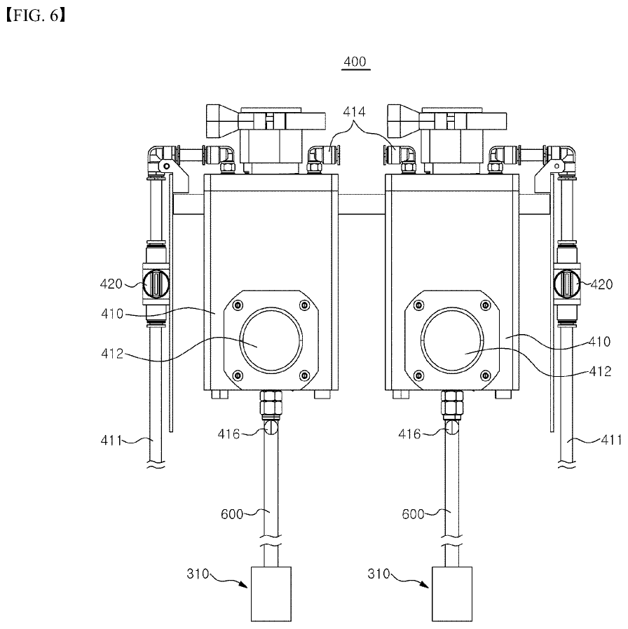

FIG. 6 is a perspective view showing the material supply device 400 according to an embodiment of the present disclosure.

Referring to FIG. 6, the material supply device 400 may include a storage tank 410 in which hydrogel is stored, and a compressor (not shown) which provides pressure to the storage tank 410.

A plurality of the storage tanks 410 may be provided, and various types of raw materials may be supplied from the respective storage tanks 410 to the respective nozzle modules 310. In this way, by providing a plurality of the storage tanks 410 and a plurality of the nozzle modules 310 corresponding thereto, a mask pack can be manufactured using various types of raw materials.

In addition, a pressure sensor (not shown) and a temperature sensor (not shown) may be provided inside the storage tank 410.

A residual pressure valve 420 is provided on the first connection pipe 411 connecting the storage tank 410 with the compressor.

The residual pressure valve 420 may be installed between the storage tank 410 and the compressor (not shown), and when the residual pressure valve 420 is opened, compressed air is supplied from the compressor to the storage tank 410, which increases the pressure inside the storage tank 410, so that the hydrogel may be moved from the storage tank 410 to the nozzle module 310 via the tube 600 to be described later. In addition, when the residual pressure valve 420 is closed, the pressure inside the storage tank 410 is discharged from an outlet 414, so that the pressure inside the storage tank 410 may be lowered to the atmospheric pressure. In this case, the opening and closing of the residual pressure valve 420 and the outlet 414 may be controlled by the control unit 500 based on the pressure measured by the pressure sensor.

In addition, a heat-resistant glass 412 made of a transparent material may be provided in the front surface of the storage tank 410. In this case, the user may determine the amount of hydrogel stored in the storage tank 410 from the outside.

Additionally, a heater (not shown) for heating the hydrogel may be provided inside or outside the storage tank 410.

The heater provided in the storage tank 410 may have a predetermined temperature range. For example, it may be a temperature range for achieving viscosity required according to the raw material stored in the storage tank 410.

In addition, the material supply device 400 may be located outside the housing 100. For example, the material supply device 400 may be located on the ceiling portion 150 or the sidewall portion of the housing 100. However, its location is not limited as long as it is located outside the housing 100. As described above, since the material supply device 400 is located outside the housing 100, even when the nozzle module 310 is in operation, the hydrogel can be supplied to the material supply device 400.

In addition, a change valve 416 may be provided between the storage tank 410, the tube 600 and the compressor.

A separate second connection pipe (not shown) may be provided to the change valve 416, so that it connects the change valve with the compressor. The change valve 416 may allow air to be directly supplied to the tube 600 from the compressor during the cleaning process of the tube 600 and the nozzle module 310, and a detailed description thereof will be presented later.

Additionally, according to an embodiment of the present disclosure, the compressor is used only to supply a raw material from the storage tank 410 to the nozzle module 310, thereby simplifying the system configuration and making maintenance easier.

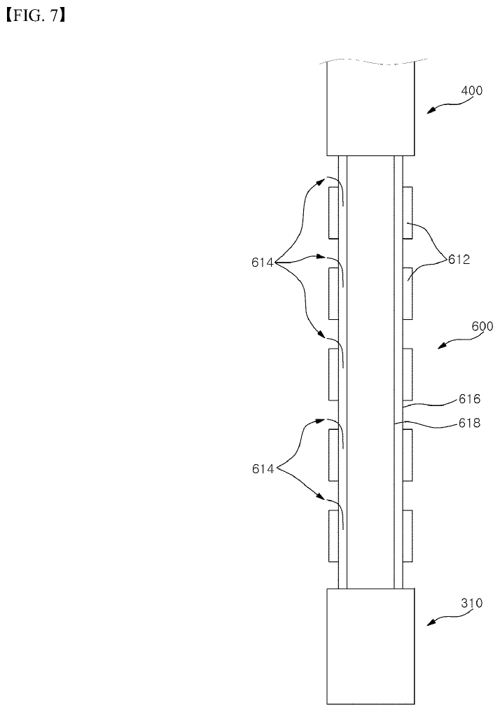

FIG. 7 is a schematic diagram briefly showing a cross section of the tube 600 connecting the material supply device 400 and the nozzle module 310 according to an embodiment of the present disclosure. The tube 600 in FIG. 7 is an exaggerated view of the outer diameter of the tube 600 for convenience of description.

The tube 600 connects the material supply device 400 with the nozzle module 310, so that the hydrogel can be transferred from the material supply device 400 to the nozzle module 310.

Between the outer circumferential surface 616 and the inner circumferential surface 618 of the tube 600, a temperature sensor 614 capable of measuring the temperature of the hydrogel moved from the material supply device 400 to the nozzle module 310 may be provided.

A plurality of temperature sensors 614 may be disposed at regular intervals along the length of the tube 600. In this case, the temperature of the hydrogel transferred through the tube 600 can be more accurately measured.

In addition, a first heating unit 612 may be provided on the outer circumferential surface 616 of the tube 600 to heat the hydrogel. In this case, the first heating unit 612 may surround the entire tube 600, or may surround a portion of the tube 600. For example, the first heating units 612 may be disposed at regular intervals along the length direction of the tube 600. Like this, by disposing the first heating units 612 at regular intervals along the length direction of the tube 600, it is possible to facilitate the motion of the tube 600 when the nozzle module 310 is moved in the X-axis or Z-axis direction.

Additionally, although not shown in the drawings, a nozzle adjusting unit may be included in the lower portion of the material supply device 400, so that the tube 600 can be wound or unwound. In this case, the nozzle adjusting unit may wind or unwind the tube 600 correspondingly to the path along which the tube 600 is moved. That is, the length of the tube 600 may be adjusted to be the same as the distance between the material supply device 400 and the nozzle module 310, so that resistance caused by the fluctuation of the tube 600 may be reduced.

The first heating unit 612 may be controlled by the control unit 500, and the temperature control range may be between 20 degrees Celsius and 90 degrees Celsius.

In addition, the tube may employ a material which has heat resistance and is non-adhesive. For example, the tube may employ Teflon. In addition, the diameter of the outer circumferential surface 616 (outer diameter) of the tube 600 may be 6 mm, and the diameter of the inner circumferential surface 618 (inner diameter) may be 4 mm.

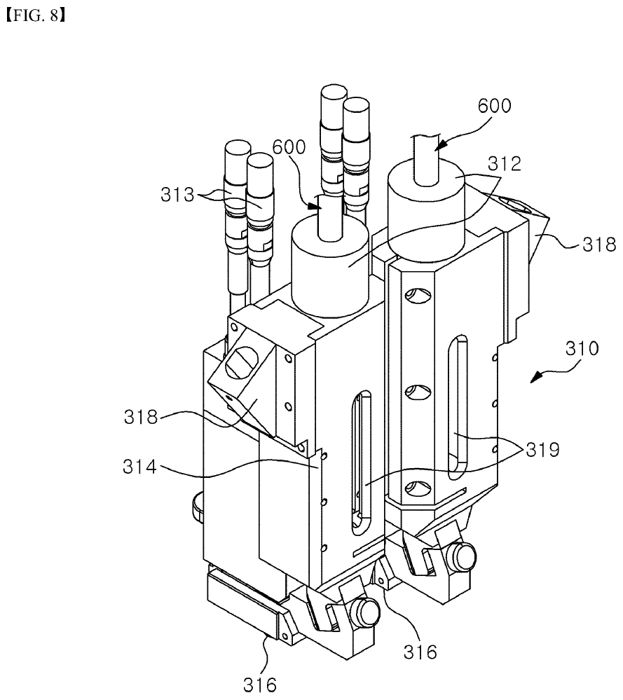

FIG. 8 is a perspective view showing the nozzle module 310 according to an embodiment of the present disclosure.

Referring to FIG. 8, the nozzle module 310 may include a cartridge 312 connected to the tube 600 and into which the hydrogel is introduced, a piezo jet spraying nozzle 316 for spraying the hydrogel stored in the cartridge 312, and a second heating unit 314 surrounding at least a portion of the cartridge 312 and the piezo jet spraying nozzle 316.

Piezoelectric element is used as the piezo jet spraying nozzle 316. In addition, the piezo jet spraying nozzle 316 has excellent responsibility since there is no difference in time between spraying the hydrogel and the electric signal applied by the control unit 500. In other words, the opening and closing time of the valve can be precisely controlled and thus has excellent precision.

The piezo jet spraying nozzle 316 is superior in printing speed and precision than a nozzle using a peristaltic pump, so it is more suitable for continuous processes and mass production.

The cartridge 312 is connected with the tube 600, and stores the hydrogel supplied from the material supply device 400. The cartridge 312 is surrounded by the second heating unit 314, so that the hydrogel therein may be heated. In addition, a hole 319 is formed in the second heating unit 314 to check the amount of hydrogel remaining in the cartridge. Additionally, a temperature sensor (not shown) may be provided in the second heating unit 314, and may be measure the temperature of the hydrogel in the cartridge 312 or the piezo jet spraying nozzle 316. Besides, the second heating unit 314 may include an introduction part 318 for introducing a wire capable of applying heat.

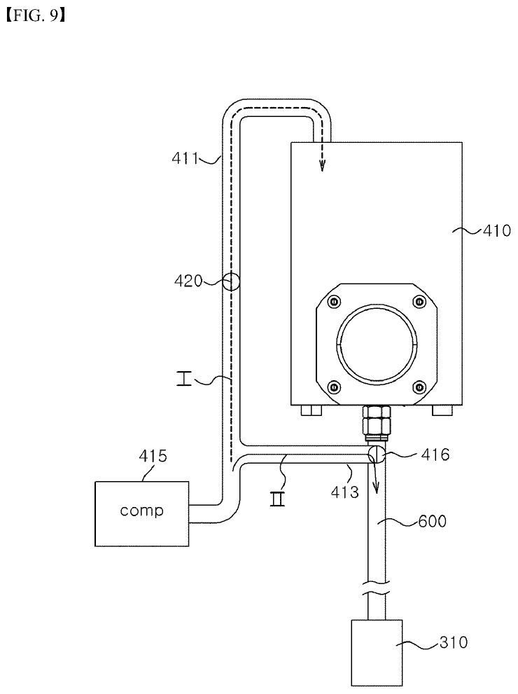

FIG. 9 is a schematic diagram showing a path through which air coming from a compressor is moved.

Although one storage tank 410 is illustrated in FIG. 9 for convenience of description, there may be a plurality of the storage tanks 410 and a plurality of the nozzle modules 310.

A path I in FIG. 9 represents a path through which air coming from the compressor is moved when the hydrogel is supplied from the storage tank 410 to the nozzle module 310. A path II represents a path through which air coming from the compressor is moved during the cleaning process of the tube 600 and the nozzle module 310.

First, the case of the path I where the hydrogel is supplied from the storage tank 410 to the nozzle module 310 will be described as follows.

When air is supplied from the compressor, the residual pressure valve 420 is opened, the change valve 416 blocks the flow of air between the second connection pipe 413 and the tube 600, and the storage tank 410 is connected with the tube 600, so that the hydrogel of the storage tank 410 flows into the tube.

Next, the cleaning process of the tube 600 and the nozzle module 310 of the path II will be described as follows.

When air is supplied from the compressor, the residual pressure valve 420 is closed, the change valve 416 allows the air to flow between the second connection pipe 413 and the tube 600, and blocks between the storage tank 410 and the tube 600. Therefore, the hydrogel stored in the storage tank 410 does not flow to the tube 600, but only the air flows to the tube 600 and the nozzle to remove the hydrogel and foreign matters remaining in the tube 600 and the nozzle module 310. Here, the residual pressure valve 420 and the change valve 416 are controlled by the control unit 500.

Additionally, the bottom portion 120 of the housing 100 may include a foreign matter collection part (not shown) capable of accommodating the hydrogel discharged from the nozzle module 310 and the foreign matters.

Hereinafter, the operation and effect of the apparatus for manufacturing a skin care pack as described above will be described.

According to an embodiment of the present disclosure, since the hydrogel can be continuously supplied to the nozzle module 310 by the material supply device 400 disposed outside the housing 100, the mask pack can be continuously mass-produced.

According to an embodiment of the present disclosure, by using the piezo jet nozzle, it is possible to increase the spraying speed of the hydrogel, and to exhibit excellent spraying precision.

According to an embodiment of the present disclosure, by providing a plurality of the storage tanks 410 and accordingly a plurality of nozzle modules 310, the mask pack can be manufactured using various types of raw materials.

According to an embodiment of the present disclosure, by providing a heater (heating unit) to the material supply device 400, the tube 600, the nozzle module 310, and the film moving module 200, the entire line through which the hydrogel is moved can be heated. Here, the heater provided to the material supply device 400 may be referred to as a third heating unit, and the heater provided to the film moving module 200 may be referred to as a fourth heating unit.

According to an embodiment of the present disclosure, by providing the film moving module 200 which is moved in the Y-axis direction independently of the X-axis and Z-axis movements, the film can be moved in response to the high-speed spraying of the hydrogel using the piezo jet nozzle.

According to an embodiment of the present disclosure, the change valve 416 is provided at a connection portion to which the storage tank 410, the tube 600, and the compressor are connected, so that cleaning of the tube 600 and the nozzle module 310 can be facilitated.

Additionally, by using the compressor to supply the raw material from the storage tank 410 to the nozzle module 310, the system configuration can be simplified and maintenance thereof can become easier.

According to an embodiment of the present disclosure, the heat-resistant glass 412 made of a transparent material may be provided on the front of the storage tank 410, so that a user can easily determine the amount of hydrogel stored in the storage tank 410 from the outside.

Followings are a list of exemplary embodiments of the present disclosure.

Section 1. An apparatus for manufacturing a skin care pack, the apparatus comprising: a housing providing a work space for forming a skin care pack; a film moving module which is provided to be movable in one direction within the work space and has a base on which a film is seated; a former which is provided to be movable in two directions perpendicular to the one direction within the work space, and includes at least one nozzle module for discharging hydrogel toward the base; and a control unit which controls the movement of the film moving module and the nozzle module, and controls the discharge of the hydrogel from the former.

Section 2. The apparatus for manufacturing a skin care pack of section 1, further comprising a material supply device for supplying the hydrogel to the nozzle module.

Section 3. The apparatus for manufacturing a skin care pack of sections 1 and 2, wherein the material supply device comprises: a storage tank in which the hydrogel is stored; and a compressor providing pressure to the storage tank to move the hydrogel stored in the storage tank to a nozzle module.

Section 4. The apparatus for manufacturing a skin care pack of sections 1 to 3, further comprising a pressure sensor provided inside the storage tank, wherein the control unit controls the pressure inside the storage tank based on pressure measured by the pressure sensor, and controls the amount of the hydrogel moved from the storage tank to the nozzle module.

Section 5. The apparatus for manufacturing a skin care pack of section 1 to 4, further comprising a tube which connects the material supply device and the nozzle module, so that the hydrogel is transferred from the material supply device to the nozzle module.

Section 6. The apparatus for manufacturing a skin care pack of sections 1 to 5, further comprising a temperature sensor which is provided between the outer circumferential surface and the inner circumferential surface of the tube to measure the temperature of the hydrogel transferred in the tube.

Section 7. The apparatus for manufacturing a skin care pack of sections 1 to 6, further comprising a first heating unit which surrounds at least a portion of the tube to heat the hydrogel that is moved from the material supply device to the nozzle module.

Section 8. The apparatus for manufacturing a skin care pack of sections 1 to 7, wherein the material of the tube is Teflon.

Section 9. The apparatus for manufacturing a skin care pack of sections 1 to 8, wherein the nozzle module comprises: a cartridge which is connected to the tube and into which a hydrogel is introduced from the tube; a piezo jet spraying nozzle which sprays the hydrogel introduced into the cartridge; and a second heating unit which surrounds at least a portion of the cartridge and the piezo jet spraying nozzle so as to heat at least a portion of the cartridge and the piezo jet spraying nozzle.

Section 10. The apparatus for manufacturing a skin care pack of sections 1 to 9, wherein a hole is formed in the second heating unit, so that the amount of the hydrogel remaining in the cartridge can be checked.

Section 11. The apparatus for manufacturing a skin care pack of sections 1 to 10, wherein the film moving module comprises: a first platform having a base on which the film is seated; a second platform which is disposed under the first platform, and has a guide part guiding the first platform to reciprocate in a Y-axis direction; and a Y-axis drive unit reciprocating the first platform in the Y-axis.

Section 12. The apparatus for manufacturing a skin care pack of sections 1 to 11, wherein the former comprises: an X-axis moving module which moves the nozzle module in an X-axis direction; and a Z-axis moving module which moves the nozzle module in a Z-axis direction.

Section 13. The apparatus for manufacturing a skin care pack of sections 1 to 12, wherein the X-axis moving module comprises: an X-axis guide plate which guides the nozzle module in the X-axis direction; a universal adapter plate supporting the X-axis guide plate; and an X-axis drive unit which is coupled to the universal adapter plate, and reciprocates the nozzle module in the X-axis.

Section 14. The apparatus for manufacturing a skin care pack of sections 1 to 13, wherein the Z-axis moving module comprises: a Z-axis guide plate guiding the nozzle module in the Z-axis direction; a support member which supports the Z-axis guide plate; and a Z-axis drive unit which is coupled to the support member, and reciprocates the nozzle module in the Z-axis.

Section 15. The apparatus for manufacturing a skin care pack of sections 1 to 14, further comprising a residual pressure valve provided on a first connection pipe connecting the storage tank with the compressor; and a change valve provided on a second connection pipe connecting the compressor with the tube, wherein the control unit controls opening and closing of the residual pressure valve and the change valve, so that air discharged from the compressor is supplied to the storage tank, or air discharged from the compressor is supplied to the tube.

While the apparatus for manufacturing a skin care pack according to examples of the disclosure has been described as concrete embodiments, these are just exemplary embodiments, and the present disclosure should be construed in a broadest scope based on the fundamental technical ideas disclosed herein, rather than as being limited to them. By combining or replacing a part or parts of embodiments disclosed herein, the ordinary skilled in the art may carry out a type of structure which is not explicitly described herein, and however, it should be noted that it shall not depart from the scope of the disclosure. Besides, the ordinary skilled in the art may easily change or modify embodiments disclosed herein based on the disclosure, and however, it is obvious that such changes or modifications also fall within the scope of the disclosure.

The present disclosure has applicability in the industrial field of a skin care pack manufacturing apparatus.

What is claimed is:

1. An apparatus for manufacturing a skin care pack, the apparatus comprising:
a housing providing a work space for forming a skin care pack;
a film moving module which is provided to be movable in one direction within the work space and has a base on which a film is seated;
a former which is provided to be movable in two directions perpendicular to the one direction within the work space, and includes at least one nozzle module for discharging hydrogel toward the base; and
a control unit which controls the movement of the film moving module and the nozzle module, and controls the discharge of the hydrogel from the former.

2. The apparatus according to claim 1, further comprising a material supply device for supplying the hydrogel to the nozzle module.

3. The apparatus according to claim 2, wherein the material supply device comprises:
a storage tank in which the hydrogel is stored; and
a compressor providing pressure to the storage tank to move the hydrogel stored in the storage tank to the nozzle module.

4. The apparatus according to claim 3, further comprising a pressure sensor provided inside the storage tank,
wherein the control unit controls the pressure inside the storage tank based on pressure measured by the pressure sensor, and controls the amount of the hydrogel moved from the storage tank to the nozzle module.

5. The apparatus according to claim 3, further comprising a tube which connects the material supply device and the nozzle module, so that the hydrogel is transferred from the material supply device to the nozzle module.

6. The apparatus according to claim 5, further comprising a temperature sensor which is provided between the outer circumferential surface and the inner circumferential surface of the tube to measure the temperature of the hydrogel transferred in the tube.

7. The apparatus according to claim 6, further comprising a first heating unit which surrounds at least a portion of the tube to heat the hydrogel that is moved from the material supply device to the nozzle module.

8. The apparatus according to claim 5, wherein the material of the tube is Teflon.

9. The apparatus according to claim 5, wherein the nozzle module comprises:
a cartridge which is connected to the tube and into which a hydrogel is introduced from the tube;
a piezo jet spraying nozzle which sprays the hydrogel introduced into the cartridge; and
a second heating unit which surrounds at least a portion of the cartridge and the piezo jet spraying nozzle so as to heat at least a portion of the cartridge and the piezo jet spraying nozzle.

10. The apparatus according to claim 9, wherein a hole is formed in the second heating unit, so that the amount of the hydrogel remaining in the cartridge can be checked.

11. The apparatus according to claim 1, wherein the film moving module comprises:
a first platform having a base on which the film is seated;
a second platform which is disposed under the first platform, and has a guide part guiding the first platform to reciprocate in a Y-axis direction; and
a Y-axis drive unit reciprocating the first platform in the Y-axis.

12. The apparatus according to claim 1, wherein the former comprises:
an X-axis moving module which moves the nozzle module in an X-axis direction; and
a Z-axis moving module which moves the nozzle module in a Z-axis direction.

13. The apparatus according to claim 12, wherein the X-axis moving module comprises:
an X-axis guide plate which guides the nozzle module in the X-axis direction;
a universal adapter plate supporting the X-axis guide plate; and
an X-axis drive unit which is coupled to the universal adapter plate, and reciprocates the nozzle module in the X-axis.

14. The apparatus according to claim 12, wherein the Z-axis moving module comprises:
a Z-axis guide plate guiding the nozzle module in the Z-axis direction;
a support member which supports the Z-axis guide plate; and
a Z-axis drive unit which is coupled to the support member, and reciprocates the nozzle module in the Z-axis.

15. The apparatus according to claim 5, further comprising:
a residual pressure valve provided on a first connection pipe connecting the storage tank with the compressor; and
a change valve provided on a second connection pipe connecting the compressor with the tube,
wherein the control unit controls opening and closing of the residual pressure valve and the change valve, so that air discharged from the compressor is supplied to the storage tank, or air discharged from the compressor is supplied to the tube.
